(19) United States
(12) Patent Application Publication
(10) Pub. No.: US 2024/0035944 A1
(43) Pub. Date: Feb. 1, 2024

(54) MEASUREMENT INSTRUMENT FOR DETERMINING ALCOHOL BY VOLUME, SPECIFIC GRAVITY, AND CALORIES OF AN ALCOHOLIC BEVERAGE

(71) Applicant: ABV Technology, Inc., Saint Paul, MN (US)

(72) Inventors: Grant Williamson, Minneapolis, MN (US); Benjamin M. Jordan, St. Paul, MN (US); Isabella M. Wenthe, Eagan, MN (US); Andrew D. LeTourneau, Stacy, MN (US)

(21) Appl. No.: 18/359,284

(22) Filed: Jul. 26, 2023

Related U.S. Application Data
(60) Provisional application No. 63/392,679, filed on Jul. 27, 2022.

(51) Int. Cl. G01N 9/36; G01N 9/26; G01N 1/44; G01N 1/42

(52) U.S. Cl. CPC G01N 9/36; G01N 9/26; G01N 1/44; G01N 1/42

(57) ABSTRACT

A process that measures alcohol by volume (ABV) of a liquid under test. The method includes determining density of the liquid using a testing device; heating or cooling the liquid for a predetermined amount of amount of time using a known amount of energy or power, and obtaining temperature measurements of the liquid during the heating or cooling; comparing the temperature measurements and the determined density to a calibrated thermal model of the liquid; and determining ABV of the liquid using the comparison to the calibrated thermal model.

### CLAIM OF PRIORITY

This application claims priority to U.S. Provisional Application Ser. No. 63/392,679, filed Jul. 27, 2022, which is incorporated by reference herein in its entirety.

### BACKGROUND

Makers of fermented beverages would like to precisely determine the alcohol by volume (ABV) of their products (e.g., to within 0.1%). Conventional approaches to measuring ABV primarily use an infrared (IR) spectroscopy method, or techniques such as ebulliometry that require a skilled lab technician. These approaches involve instrumentation and skill sets that may be too costly for smaller fermented beverage producers. The present inventors have recognized a need for an improved approach for accurate measurement of the ABV of beverages.

### OVERVIEW

This document relates generally to techniques of measuring the ABV of fermented beverages.

A method example includes determining density of the liquid using the testing device, heating or cooling the liquid for a predetermined amount of time using a predetermined amount or measured amount of energy or power, obtaining temperature measurements of the liquid during the heating or cooling, comparing the temperature measurements and the determined density to a calibrated thermal model of the liquid, and determining ABV of the liquid using the comparison to the calibrated thermal model.

An apparatus example includes a density measurement chamber including a pressure sensor that produces a differential pressure measurement of a liquid under test, a heating and cooling block including a sample chamber and one or more temperature sensors that produce a temperature measurement of a sample of the liquid under test in the sample chamber, and a logic unit including logic circuitry. The logic unit is configured to initiate a non-boiling heating or cooling cycle of the sample liquid in the sample chamber for a predetermined amount of time using a predetermined amount of energy or power, obtain temperature measurements of the sample liquid during the heating or cooling cycle, produce a value of density of the liquid under test using the differential pressure measurement, compare the temperature measurements and the value of density to a calibrated thermal model of the sample liquid, and produce a value of ABV of the sample liquid using the comparison to the calibrated thermal mode.

A control system example includes a pressure sensor and one or more temperature sensors, a heating element, and a logic unit operatively coupled to the pressure sensor and the one or more temperature sensors. The logic unit includes logic circuitry and is configured to receive a value of differential pressure of the liquid from the pressure sensor and compute a value of density of the liquid, control the heating element to apply a non-boiling heating or cooling cycle to the liquid that consumes a predetermined amount of energy, receive values of temperature of the liquid during the heating or cooling cycle from the one or more temperature sensors, compare the temperature measurements and the value of density to a calibration thermal model of the liquid, and produce a value of ABV of the liquid using the comparison to the calibrated thermal model.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a flow diagram of an example of a method of automatic measurement of alcohol by volume (ABV) of a liquid under test.

FIG. 2 is a block diagram of flow of the liquid under test in an example testing device.

FIG. 3 is a side view of an example of a density measurement chamber.

FIG. 4 is a top view of an example of a density measurement chamber.

FIG. 5 is an example of a testing device having a heating and cooling block.

FIG. 6 is a top view of an example of a heating and cooling block.

FIG. 7 is a block diagram of an example of a control system for a testing device to measure ABV of a liquid under test.

FIG. 8 is a block diagram of a method for determining the ABV of a liquid.

### DETAILED DESCRIPTION

As explained previously herein, makers of fermented beverages would like to precisely determine the alcohol by volume (ABV) of their products. Smaller producers may consider the instrumentation needed for conventional methods (e.g., IR spectroscopy) too costly. Ebulliometry is another method, but this technique places restrictions on the liquid under test (e.g., carbon dioxide level) and requires a skilled lab technician. An improved approach measures the thermal properties and density of a sample of the fermented beverage to determine ABV. This technique can be performed with instruments that are lower cost than conventional approaches. Specific gravity and the caloric content of the fermented beverage can also be determined.

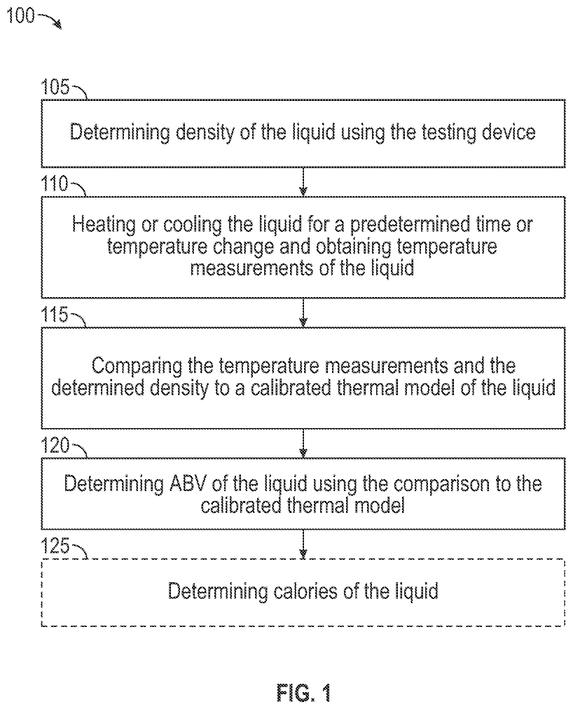

FIG. 1 is a flow diagram of an example of a method 100 of automatic measurement of ABV of a liquid under test. The liquid under test can be a fermented beverage or any liquid with three primary non-dilute components (e.g., including ethanol, water, and sugar). The method 100 is implemented using a testing device. At block 105, the specific gravity or density of the liquid is determined using the testing device. Thermal properties of the liquid are determined after the density measurement. At block 110, the liquid is heated or cooled using a known amount of energy (e.g., electrical energy) or power. The known amount of energy may be a measured amount of energy that may vary, or a predetermined amount of energy applied during the testing. The heating or cooling may be for a predetermined time at the known power, the heating or cooling may be for a predetermined temperature change at a predetermined power, or the heating and cooling may be to a setpoint temperature at a predetermined power.

At block 115, the temperature measurements and the determined density are compared to a calibrated thermal model of the liquid. The calibrated model may be stored in memory of the device. At block 120, a logic unit compares the temperature change in the liquid, the power or energy applied to the liquid, and the density of the liquid to the calibrated thermal model to determine the ABV. Multiple cycles (or runs) of one or both of heating and cooling may be performed and averaged to improve the accuracy of the determined ABV.

Optionally, calories of the liquid are also determined at block 125 using the computed density. Any combination of ABV, specific gravity, and calories may be presented to the user at the conclusion of the automatic measurement by the testing device.

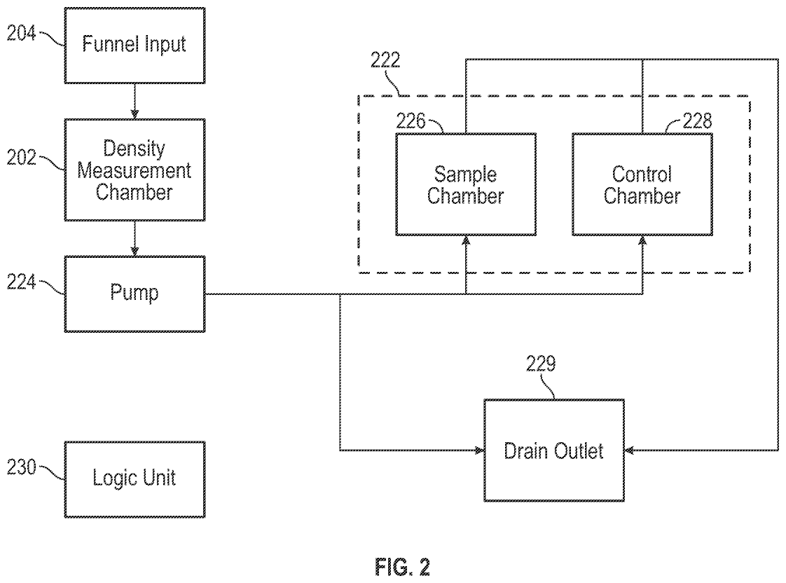

FIG. 2 is a block diagram of flow of the liquid under test in an example testing device. A sample of the liquid under test is fed into a density measurement chamber 202 of the testing device. The testing device may have a funnel input 204 to the density measurement chamber 202 to receive the liquid sample.

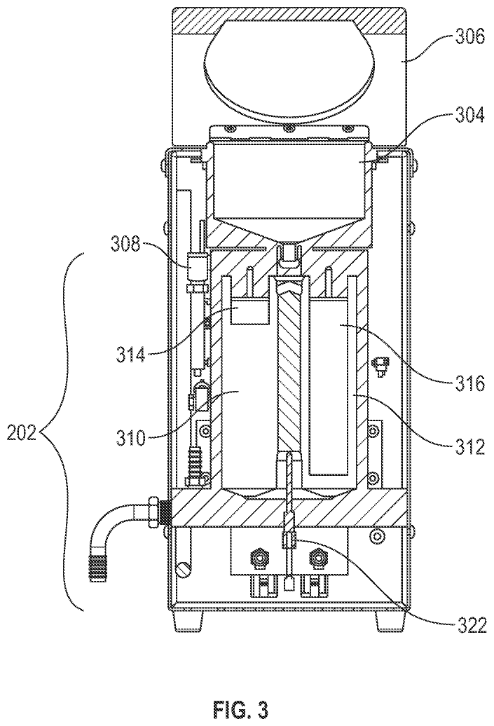

FIG. 3 is an example of a testing device that includes a density measurement chamber 202. The testing device includes a fill container 304 and a lid 306. The fill container 304 holds a filter (not shown) that may or may not be removable. An operator adds a sample of the liquid to the fill container 304 that may include the funnel input 204 of FIG. 2. The volume of the sample for the density measurement can be small (e.g., ten ounces (10 oz.)). The sample liquid may gravity drain into the density measurement chamber 202. The sample liquid may be directed into the density measurement chamber 202 using a tube, flow chamber, or packing material. In some examples, a pump is used to move the sample liquid from the fill container 304 to the density measurement chamber 202.

The density measurement chamber 202 includes a pressure sensor that provides an electrical signal representative of pressure of the sample liquid in the density measurement chamber 202. In the example of FIG. 3, the pressure sensor is a differential pressure sensor that measures a differential pressure of the liquid in the chamber. The density measurement chamber 202 includes two columns, a low pressure column 310 and a high pressure column 312. A temperature probe 322 monitors temperature of the liquid. The low pressure column includes a low pressure tube 314 and the high pressure column includes a high pressure tube 316. The diameter of the tubes is sized (e.g., 1 inch) such that the liquid can drain freely without being inhibited by surface tension on the tubes or capillary action at the edges of the tubes. A pressure measurement may be obtained when the density measurement chamber 202 has filled to overflow out the overflow line 308, or has filled to above the low pressure tube 314.

At the top or near the top of the low pressure tube 314 is a low pressure port to the differential pressure sensor, and at the top or near the top of the high pressure tube 316 is a high pressure port to the differential pressure sensor. The high pressure tube 316 extends a greater depth into the liquid than the low pressure tube 314 such that there is a distance, or difference in height (Δh), between the liquid level in the high pressure tube and the low pressure tube based on the pressure of the liquid column. In an example, the distance between the low pressure port and the high pressure port is 4 inches, but the distance between ports can be in the range of greater than zero inches to about 24 inches.

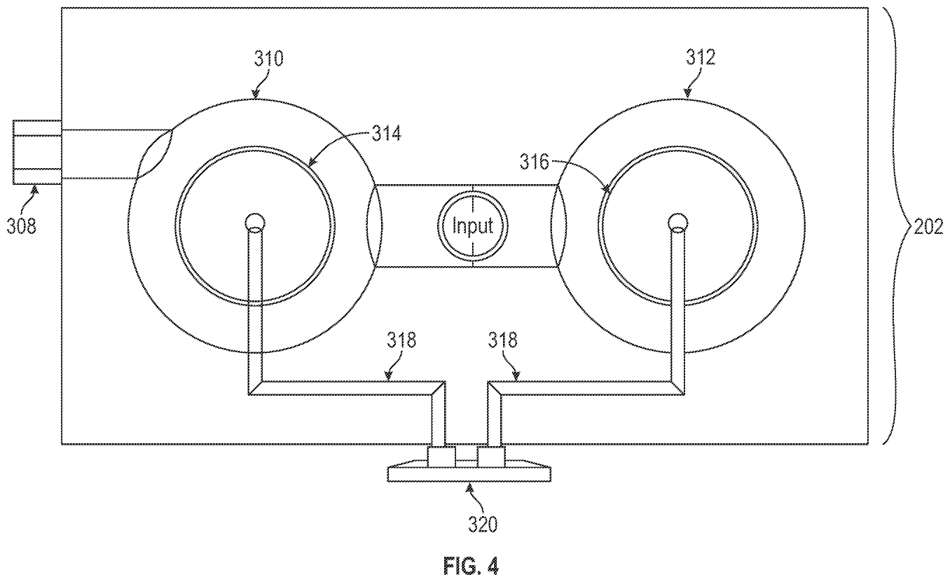

FIG. 4 is a top view of the density measurement chamber 202. FIG. 4 shows a top view of the overflow line 308, the low pressure column 310, the low pressure tube 314, the high pressure column 312, and the high pressure tube 316. The low pressure port and the high pressure port of the tubes are connected to pressure sense lines 318 that run to the differential pressure sensor 320.

The differential pressure measurement from the differential pressure sensor is used to calculate density of the liquid under test. The differential pressure measurement may be provided to the logic unit 230 in FIG. 2 that includes a microprocessor to compute the density. The density can be computed by the logic unit 230 from

P=ρgh,

where P is the differential pressure, g is the gravity constant, h is the difference in height in the liquid between the low pressure port and the high pressure port, and p is the density. The logic unit 230 may also compute the temperature adjusted specific gravity of the liquid by dividing the computed temperature adjusted density by the density of water at a specific temperature. The logic unit 230 may perform multiple density measurements at various points in a run (e.g., between heating and cooling) to improve accuracy or precision of the determined density value.

The differential pressure sensor is very precise (e.g., 0.001 grams per cubic centimeter (0.001 g/cm³) accuracy and repeatability for density). A calibration procedure may be used to re-reference the pressure sensor readings for manufacturing differences or for drift occurring in the readings. The calibration procedure may determine a constant A to use for the density calculation such that

P=A(ρgh)

Temperature probe 322 provides a temperature reading of the liquid to compensate the density and pressure to a specific liquid temperature. The density reading may also be adjusted based on one or more of vapor pressure, atmospheric pressure, or temperature.

In the example of FIG. 2, when one or both of the density and the specific gravity are computed by the logic unit 230, the liquid sample is moved to a heating and cooling block 222. A pump 224 may be used to move the liquid sample from the density measurement chamber 202 to the heating and cooling block 222, or the liquid sample may be gravity fed into the heating and cooling block 222. The chambers are emptied using the drain outlet 229.

The heating and cooling block 222 includes one or more sample chambers 226 and may contain a control chamber 228. The sample chambers 226 hold the sample liquid moved from the density measurement chamber 202. The control chamber 228 holds a control liquid (e.g., deionized water). The heating and cooling block 222 also includes temperature sensors (e.g., thermistors) that collect temperature measurements of the sample liquid and the control liquid, and the temperature at various locations of the block 222.

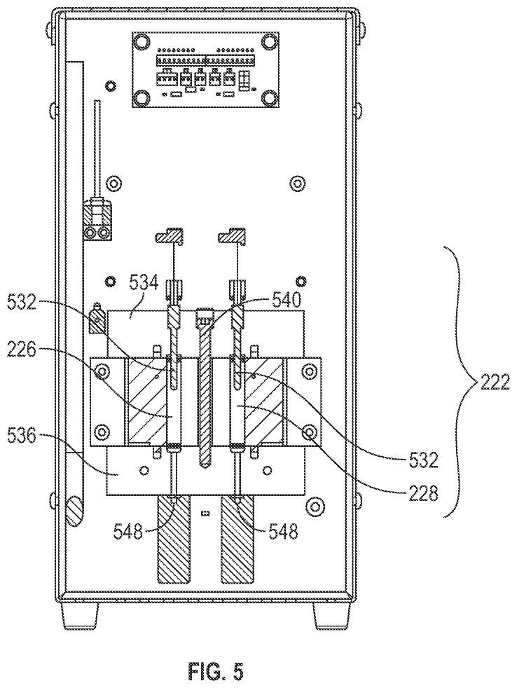

FIG. 5 is an example of a testing device having a heating and cooling block 222 that includes one or more sample chambers 226 and a control chamber 228. In some examples, the testing device includes only one sample chamber 226 and may or may not include a control chamber 228. Each chamber includes a liquid temperature probe 532 having one or more temperature sensors (e.g., thermistors). Temperature sensors are also disposed at various locations of the heating and cooling block 222. The heating and cooling block 222 includes a top manifold 534 and a bottom manifold 536. Each of the manifolds has low thermal conductivity. The manifolds may include valves that change position to control flow. The heating and cooling block 222 may be made of aluminum, and the heating and cooling block 222 may be covered with thermal insulation. There may also be an air gap 540 between the sample chamber 226 and the control chamber 228. The sample chamber 226 and control chamber 228 are symmetrically placed in the heating and cooling block 222. The chambers may hold the same volume of liquid (e.g., one milliliter or 1 ml). The chambers may be filled from the bottom 548 or filled from the top.

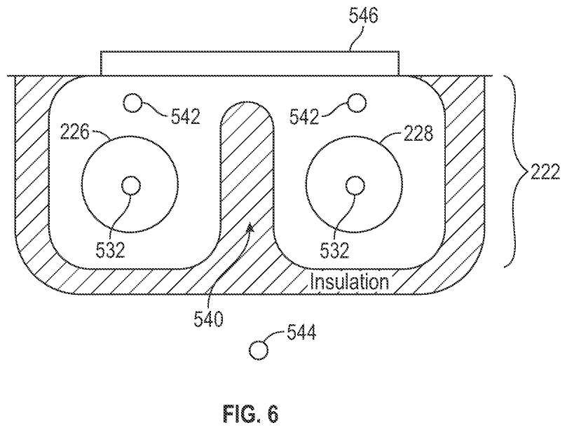

FIG. 6 is a top view of the heating and cooling block 222, showing the sample chamber 226, control chamber 228, liquid temperature probes 532 and the air gap 540. Also shown are block temperature probes 542, an ambient temperature probe 544, and a heater/cooler 546. The heater/cooler 546 can be separate heating and cooling devices or a combination device, such as a thermoelectric peltier.

To determine ABV, the heating and cooling block 222 is heated or cooled, or both heated and cooled. The thermal response of the sample liquid and the control liquid is determined using the temperature probes. Temperature measurements are collected for the sample liquid, the control liquid, the temperature probe 542 near the sample chamber 226, the temperature probe 542 near the control chamber 228, and the ambient temperature probe 544. Temperature measurements are collected during one or both of the heating and cooling of the liquids. The temperature measurements of the heating and cooling cycle can be included in the thermal response of the sample liquid and the control liquid.

The energy applied to the heating and cooling block 222 is tracked. The energy can include electrical energy and can be tracked by monitoring one or more of the power, voltage, and current delivered to the heater/cooler 546. The collected temperature data, the amount of energy applied to the heater/cooler 546, and the computed density are then applied to a calibrated thermal model by the logic unit 230. In some examples, the calibrated thermal model is a statistical model of the thermal response to one or both of heating and cooling.

An example of a procedure for determining ABV using a statistical model of the thermal response is as follows. The temperature data from at least one heating or cooling cycle (or run) is analyzed using a comparison to a calibration data set. For a moving average of a data set of the temperature data points (e.g., a 50 data point forward-looking moving average), the temperature difference between the sample chamber side and the control chamber side of the heating and cooling block 222 is calculated for a given time window of the data set (e.g., a 0 to 60 second time window). The data is then time-shifted such that the starting block temperature difference is the same for this data set and all data within the calibration data set. For a moving average of a set of the data points (e.g., the 50 datapoint forward-looking moving average), the temperature difference between the liquid temperature on the sample chamber side and the liquid temperature on the control chamber side is calculated for the time-adjusted dataset. Then the block temperature differences are subtracted from the liquid temperature differences at each time-adjusted time point. The values of this difference are then summed (time duration between each time point is typically the same for each dataset including both the calibration data set and the analyzed run temperature data, but in case they are not the sum can be a time-weighted sum). This sum is then compared to the calibration data set along with the density and in some cases the ambient temperature and together this comparison provides the ABV. Typically to achieve the maximum ABV resolution for this statistical approach, the run temperature data under analysis starts off substantially equilibrated in temperature, including with ambient and only consists of a single heating or cooling step. The ABV results of multiple heating or cooling steps can be averaged together to improve the precision and accuracy of the ABV measurement.

The heating and the cooling cycles are initiated by the logic unit 230. The logic unit 230 may initiate multiple heating and cooling cycles and apply the temperature measurements from the multiple cycles to the calibration model to improve the accuracy of the computations.

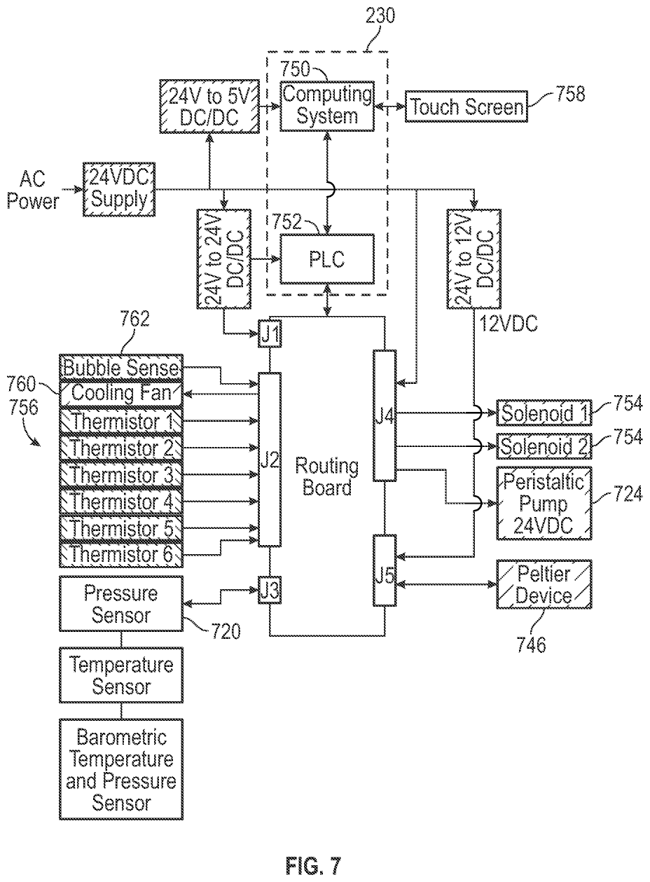

FIG. 7 is a block diagram of an example of a control system for a testing device to measure ABV. The control system includes a logic unit 230 that includes a computing system 750 and a programmable logic controller (PLC) 752. The computing system 750 can include a processor (e.g., a microprocessor) and memory. The processor executes machine instructions stored in the memory to perform the functions described. The PLC 752 includes control logic circuitry for controlling the flow of the sample liquid and the heating and cooling of the heating and cooling block 222. The PLC 752 may implement a state machine or logic sequencer to advance through logic steps to activate one or more portions of the testing device to perform the functions described. The PLC 752 activates solenoids 754 to open and close valves and may activate pump 724 to move the sample liquid and the control liquid in the testing device, such as from the density measurement chamber 202 to the sample chamber 226 of the heating and cooling block 222. Heating and cooling are provided by the peltier 746A cooling fan 760 can be included to blow air by the peltier 746. The air can be hotter or cooler than the peltier 746. The bubble sense is a sensor 762 that can be included to sense the presence of liquid in the drain line.

The computing system 750 receives pressure information from pressure sensor 720 and receives temperature information from temperature sensors 756. The temperature sensors 756 provide temperature information for the liquid temperature probes, the block temperature probes, and the ambient temperature probe or probes. Using the pressure information and relevant temperature information, the computing system 750 calculates one or both of density and specific gravity. The computing system 750 applies the temperature information, the energy information of the energy applied to the heater/cooler, and the calculated density to the calibration model to determine a value of ABV of the sample liquid. The computing system 750 may determine a value of caloric content of the liquid using the calculated specific gravity value and ABV value. A user interface including a touchscreen display 758 is operatively coupled to the computing system 750. The computing system 750 may present prompts for the user to perform and may present the results of the measurements.

In some examples, the calibrated thermal model is a heat transfer model for heat transfer of the heating and cooling block 222. The heat transfer model may use the following equations:

dTa/dt=alpha_ab

dQ/dt=−b_q*x[1]+K_q*Ta,

dTcb/dt=alpha_ccb*(Tc−Tcb)+kappa_cb*Q,

dTsb/dt=alpha_ssb*(Ts−Tsb)+kappa_sb*Q,

dTc/dt=alpha_ccb*(Tcb−Ts), and

dTs/dt=alpha_ssb*(Tsb−Ts),

where:
Ta is the ambient temperature,
alpha_ab is the heat transfer constant heat transfer constant between the heating and cooling block 222 and ambient,
Q is the heat transfer into the heating and cooling block 222,
b_q is a constant to account for units from power applied to the peltier 746 to heat transfer into the heating and cooling block 222,
x[1] is the power to the peltier 746,
K_q is a constant to account for changes in heat transfer into the heating and cooling block 222 due to ambient conditions,
Tcb is the temperature of the control chamber 228,
alpha_ccb is the heat transfer constant between the control chamber 228 and the control liquid,
Tc is the temperature of the control liquid,
kappa_cb is a constant used to adjust heat transfer into the control chamber 228,
Tsb is the temperature of the sample chamber 226,
apha_ssb is the heat transfer constant between the sample chamber 226 and the sample liquid,
Ts is the temperature of the sample liquid, and
kappa_sb is a constant used to adjust heat transfer into the sample chamber 226.

Sensor information and power information is collected and compared to the numerical solution for the heat transfer model to determine the composition of the sample liquid.

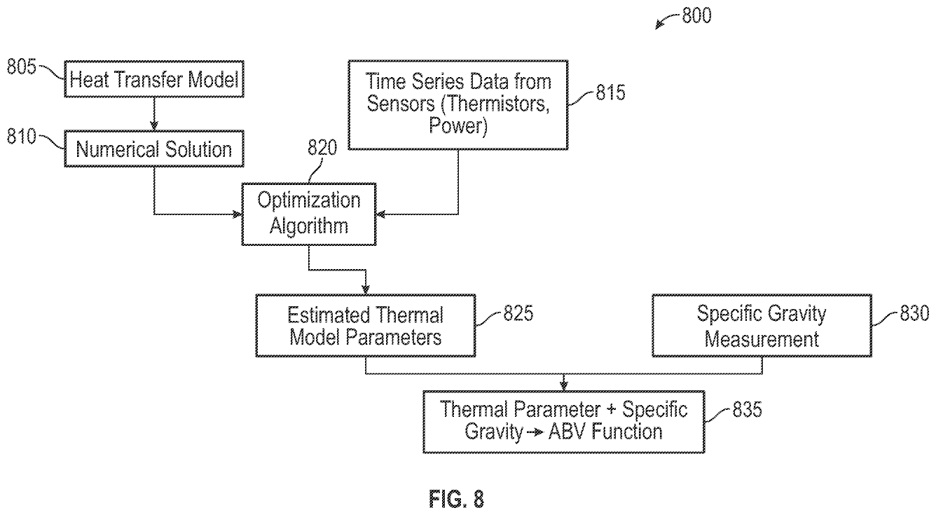

FIG. 8 is a block diagram of a method 800 for determining the ABV of a liquid. At block 805, the heat transfer model may use the equations described previously herein to produce a numerical solution at block 810. At block 820, the numerical solution of block 810 and a time series of data collected from the sensors at block 815 are optimized. The optimized solution from block 820 is used at block 825 to estimate parameters of the calibrated thermal model. In some examples, the calibrated thermal model includes a combination of a heat transfer model and a statistical model, and the combination is used to produce the parameters. At block 830 the specific gravity is measured. At block 835, the estimated thermal model parameters of block 825 and the specific gravity measurement of block 830 are used to produce the ABV function. One or any combination of ABV, specific gravity, and calories of the liquid are presented to the user.

The methods, devices, and systems described herein automatically measure the ABV of a liquid (e.g., a fermented malt beverage) and present the result to a user. The techniques described can determine ABV of a liquid under test that is between 0.1% and 20% alcohol by volume with 0.1% ABV accuracy and repeatability. Accurate results can be provided in less than fifteen minutes. The testing device can determine density of the liquid under test that has a density between 0.95 to 1.2 grams per cubic centimeter (g/cm³) with up to 0.001 g/cm³ accuracy and repeatability.

### ADDITIONAL DESCRIPTION AND EXAMPLES

Example 1 includes subject matter (such as a method of operating a testing device to automatically measure alcohol by volume (ABV) of a liquid under test) comprising determining density of the liquid using the testing device; heating or cooling the liquid for a predetermined amount of time using a known amount of energy or power, or heating or cooling the liquid to cause a predetermined temperature change using the known amount of energy or power; obtaining temperature measurements of the liquid during the heating or cooling; comparing the temperature measurements and the determined density to a calibrated thermal model of the liquid; and determining ABV of the liquid using the comparison to the calibrated thermal model.
In Example 2, the subject matter of Example 1 optionally includes heating or cooling a sample of the liquid in a sample chamber of a heating and cooling block of the device and heating or cooling a control sample in a control chamber of the heating and cooling block for the predetermined amount of time or for the predetermined temperature change using the known amount of energy or power; comparing the temperature measurements and the determined density to a heat transfer model of the heating and cooling block; and determining ABV of the sample liquid using the comparison to the heat transfer model as the calibrated thermal model.
In Example 3, the subject matter of one or both of Examples 1 and 2 optionally includes heating or cooling a sample of the liquid in a sample chamber of a heating and cooling block of the device and heating or cooling a control sample in a control chamber of the heating and cooling block for the predetermined amount of time or for the predetermined temperature change using the known amount of energy or power; comparing the determined density and temperature measurements of the sample liquid, the control liquid, the heating and cooling block, and ambient. to a statistical thermal model of the sample liquid and heating and cooling block; and determining ABV of the liquid using the comparison to the statistical thermal model as the calibrated thermal model.
In Example 4, the subject matter of one or any combination of Examples 1-3 optionally includes multiple cycles of heating and cooling of both a sample of the liquid under test and a control sample of a control liquid in a heating and cooling block for the predetermined amount of time or for the predetermined temperature change using the known amount of energy or power, and obtaining temperature measurements of the sample liquid, the control liquid, the heating and cooling block, and ambient, during the multiple cycles of heating and cooling.
In Example 5, the subject matter of one or any combination of Examples 1˜4 optionally includes measuring a differential pressure of the liquid using a differential pressure sensor, and calculating a value of the density of the liquid using the differential pressure measurement.
In Example 6, the subject matter of Example 5 optionally includes extracting a sample of the liquid, and measuring a pressure difference between pressure at a first depth of the sample and pressure at a second depth of the sample liquid. The first depth and the second depth are separated by between zero and 24 inches.
In Example 7, the subject matter of one or any combination of Examples 1-6 optionally includes measuring a differential pressure of the liquid using a differential pressure sensor, measuring temperature of the liquid, calculating a value of specific gravity of the liquid using the differential pressure measurement and the temperature measurement, and determining a value of caloric content of the liquid using the calculated specific gravity value and the determined ABV.
In Example 8, the subject matter of one or any combination of Examples 1-7 optionally includes the liquid under test being a liquid that comprises three primary non-dilute components.
In Example 9, the subject matter of one or any combination of Examples 1-7 optionally includes the liquid under test being a fermented beverage.
Example 10 includes subject matter (such as an apparatus) or can optionally be combined with one or any combination of Examples 1-9 to include such subject matter, comprising a density measurement chamber including a pressure sensor that produces a differential pressure measurement of a liquid under test, a heating and cooling block including a sample chamber and one or more temperature sensors that produce a temperature measurement of a sample of the liquid under test in the sample chamber, and a logic unit. The logic unit including logic circuitry configured to initiate a non-boiling heating or cooling cycle of the sample liquid in the sample chamber for a predetermined time using a known amount of energy or power, or heating or cooling the liquid to cause a predetermined temperature change using the known amount of energy or power, obtain temperature measurements of the sample liquid during the heating cycle, produce a value of density of the liquid under test using the differential pressure measurement, compare the temperature measurements and the value of density to a calibrated thermal model of the sample liquid, and produce a value of alcohol by volume (ABV) of the sample liquid using the comparison to the calibrated thermal model.
In Example 11, the subject matter of Example 10 optionally includes a heating and cooling block that includes a control chamber and one or more temperature sensors that produce a temperature measurement of a control liquid in the sample chamber; a logic unit is configured to initiate one or more heating and cooling cycles of the sample liquid in the sample chamber and the control liquid in the control chamber and obtain temperature measurements of the sample liquid and control liquid during the one or more heating and cooling cycles; and compare the temperature measurements and the determined density to a heat transfer model of the heating and cooling block as the calibrated thermal model.
In Example 12, the subject matter of Example 11 optionally includes a heating and cooling block includes an air gap arranged between the sample chamber and the control chamber.
In Example 13, the subject matter of one or any combination of Examples 10-12 optionally includes a heating and cooling block that includes a control chamber and one or more temperature sensors that produce a temperature measurement of a control liquid in the sample chamber; and a logic unit configured to initiate one or more heating and cooling cycles of the sample liquid in the sample chamber and the control liquid in the control chamber and obtain temperature measurements of the sample liquid and control liquid during the one or more heating and cooling cycles, and compare the temperature measurements and the determined density to a statistical thermal model of the sample liquid and heating and cooling block as the calibrated thermal model.
In Example 14, the subject matter of one or any combination of Examples 10-13 optionally includes a pressure sensor that includes a lower pressure port and a higher pressure port, and the higher pressure port is separated from the lower pressure port by between zero and 24 inches.
In Example 15, the subject matter of one or any combination of Examples 10-14 optionally includes a pressure sensor that includes two tubes each respectively connected to one of the lower pressure port and the higher pressure port, and wherein the diameter of the tubes is large enough for the liquid under test to drain from the tubes without the draining being inhibited by surface tension or capillary forces.
In Example 16, the subject matter of one or any combination of Examples 10-15 optionally includes density measurement chamber includes a temperature sensor that produces a temperature measurement of the liquid under test; and a logic unit configured to calculate a value of specific gravity of the liquid using the using the differential pressure measurement and the temperature measurement, and determine a value of caloric content of the liquid using the calculated specific gravity value and the value of ABV.
In Example 17, the subject matter of on or any combination of Examples 10-16 optionally includes a logic unit is configured to produce a value of ABV of a liquid under test that includes three primary non-dilute components one of which is ethanol.
In Example 18, the subject matter of one or any combination of Examples 10-17 optionally includes a pump to move at least a portion of liquid in the density measurement chamber to the sample chamber of the heating and cooling block.
Example 19 includes subject matter (such as a control system for an apparatus that produces a value of alcohol by volume (ABV) of a liquid under test) or can optionally be combined with one or any combination of Examples 1-18 to include such subject matter, comprising a pressure sensor and one or more temperature sensors, a heating element, and a logic unit operatively coupled to the pressure sensor and the one or more temperature sensors and including logic circuitry. The logic unit is configured to receive a value of differential pressure of the liquid from the pressure sensor and compute a value of density of the liquid, control the heating element to apply a non-boiling heating or cooling cycle to the liquid that consumes a known amount of energy or power, receive values of temperature of the liquid during the heating or cooling cycle from the one or more temperature sensors, compare the temperature measurements and the value of density to a calibrated thermal model of the liquid, and produce a value of ABV of the liquid using the comparison to the calibrated thermal model.
In Example 20, the subject matter of Example 19 optionally includes a cooling device; and a logic unit configured to initiate one or more non-boiling heating and cooling cycles of a control liquid and the liquid under test; receive values of temperature of the control liquid, the liquid under test, and a heating and cooling block containing the control liquid and liquid under test, from the one or more temperature sensors during the one or more heating and cooling cycles; and compare the computed value of density and the temperature values of the control liquid, the liquid under test, and the heating and cooling block, to the calibrated thermal model of the liquid.
In Example 21, the subject matter of Example 20 optionally includes a logic unit configured to compare the temperature measurements and the value of density to a heat transfer model of the heating and cooling block as the calibrated thermal model, and produce a value of ABV of the liquid using the comparison to the heat transfer model.

The non-limiting Examples can be combined in any permutation or combination. The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. Method examples described herein can be machine or computer-implemented at least in part.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method of operating a testing device to automatically measure alcohol by volume (ABV) of a liquid under test, the method comprising:
determining density of the liquid using the testing device;
heating or cooling the liquid for a predetermined amount of time using a known amount of energy or power, or heating or cooling the liquid to cause a predetermined temperature change using the known amount of energy or power;
obtaining temperature measurements of the liquid during the heating or cooling;
comparing the temperature measurements and the determined density to a calibrated thermal model of the liquid; and
determining ABV of the liquid using the comparison to the calibrated thermal model.

2. The method of claim 1, including:
wherein the heating or cooling the liquid includes heating or cooling a sample of the liquid in a sample chamber of a heating and cooling block of the device and heating or cooling a control sample in a control chamber of the heating and cooling block for the predetermined amount of time or for the predetermined temperature change using the known amount of energy or power;
wherein the comparing to a calibrated model includes comparing the temperature measurements and the determined density to a heat transfer model of the heating and cooling block; and
wherein the determining ABV includes determining ABV of the sample liquid using the comparison to the heat transfer model as the calibrated thermal model.

3. The method of claim 1, including:
wherein the heating or cooling the liquid includes heating or cooling a sample of the liquid in a sample chamber of a heating and cooling block of the device and heating or cooling a control sample in a control chamber of the heating and cooling block for the predetermined amount of time or for the predetermined temperature change using the known amount of energy or power;
wherein the comparing to a calibrated model includes comparing the determined density and temperature measurements of the sample liquid, the control liquid, the heating and cooling block, and ambient. to a statistical thermal model of the sample liquid and heating and cooling block; and
wherein the determining ABV includes determining ABV of the liquid using the comparison to the statistical thermal model as the calibrated thermal model.

4. The method of claim 1, wherein the heating or cooling the liquid includes multiple cycles of heating and cooling of both a sample of the liquid under test and a control sample of a control liquid in a heating and cooling block for the predetermined amount of time or for the predetermined temperature change using the known amount of energy or power, and obtaining temperature measurements of the sample liquid, the control liquid, the heating and cooling block, and ambient, during the multiple cycles of heating and cooling.

5. The method of claim 1, wherein determining the density of the liquid includes:
measuring a differential pressure of the liquid using a differential pressure sensor; and
calculating a value of the density of the liquid using the differential pressure measurement.

6. The method of claim 5, wherein measuring the differential pressure includes:
extracting a sample of the liquid; and
measuring a pressure difference between pressure at a first depth of the sample and pressure at a second depth of the sample liquid, wherein the first depth and the second depth are separated by between zero and 24 inches.

7. The method of claim 1, including:
measuring a differential pressure of the liquid using a differential pressure sensor;
measuring temperature of the liquid;
calculating a value of specific gravity of the liquid using the differential pressure measurement and the temperature measurement; and
determining a value of caloric content of the liquid using the calculated specific gravity value and the determined ABV.

8. The method of claim 1, wherein the liquid under test comprises three primary non-dilute components.

9. The method of claim 8, wherein the liquid under test is a fermented beverage.

10. An apparatus comprising:
a density measurement chamber including a pressure sensor that produces a differential pressure measurement of a liquid under test;
a heating and cooling block including a sample chamber and one or more temperature sensors that produce a temperature measurement of a sample of the liquid under test in the sample chamber; and
a logic unit including logic circuitry, the logic unit configured to:
initiate a non-boiling heating or cooling cycle of the sample liquid in the sample chamber for a predetermined time using a known amount of energy or power, or heating or cooling the liquid to cause a predetermined temperature change using the known amount of energy or power;
obtain temperature measurements of the sample liquid during the heating cycle;
produce a value of density of the liquid under test using the differential pressure measurement;
compare the temperature measurements and the value of density to a calibrated thermal model of the sample liquid; and
produce a value of alcohol by volume (ABV) of the sample liquid using the comparison to the calibrated thermal model.

11. The apparatus of claim 10,
wherein the heating and cooling block includes a control chamber and one or more temperature sensors that produce a temperature measurement of a control liquid in the sample chamber;
wherein the logic unit is configured to initiate one or more heating and cooling cycles of the sample liquid in the sample chamber and the control liquid in the control chamber and obtain temperature measurements of the sample liquid and control liquid during the one or more heating and cooling cycles; and
wherein the logic unit is configured to compare the temperature measurements and the determined density to a heat transfer model of the heating and cooling block as the calibrated thermal model.

12. The apparatus of claim 11, wherein the heating and cooling block includes an air gap arranged between the sample chamber and the control chamber.

13. The apparatus of claim 10,
wherein the heating and cooling block includes a control chamber and one or more temperature sensors that produce a temperature measurement of a control liquid in the sample chamber;
wherein the logic unit is configured to initiate one or more heating and cooling cycles of the sample liquid in the sample chamber and the control liquid in the control chamber and obtain temperature measurements of the sample liquid and control liquid during the one or more heating and cooling cycles; and
wherein the logic unit is configured to compare the temperature measurements and the determined density to a statistical thermal model of the sample liquid and heating and cooling block as the calibrated thermal model.

14. The apparatus of claim 10, wherein the pressure sensor of the density measurement chamber includes a lower pressure port and a higher pressure port, and the higher pressure port is separated from the lower pressure port by between zero and 24 inches.

15. The apparatus of claim 10,
wherein the pressure sensor of the density measurement chamber includes two tubes each respectively connected to one of the lower pressure port and the higher pressure port, and wherein the diameter of the tubes is large enough for the liquid under test to drain from the tubes without the draining being inhibited by surface tension or capillary forces.

16. The apparatus of claim 10,
wherein the density measurement chamber includes a temperature sensor that produces a temperature measurement of the liquid under test;
wherein the logic unit is configured to:
calculate a value of specific gravity of the liquid using the using the differential pressure measurement and the temperature measurement; and
determine a value of caloric content of the liquid using the calculated specific gravity value and the value of ABV.

17. The apparatus of claim 10, wherein the logic unit is configured to produce a value of ABV of a liquid under test that includes three primary non-dilute components one of which is ethanol.

18. The apparatus of claim 10, including a pump to move at least a portion of liquid in the density measurement chamber to the sample chamber of the heating and cooling block.

19. A control system for an apparatus that produces a value of alcohol by volume (ABV) of a liquid under test, the control system including:
a pressure sensor and one or more temperature sensors;
a heating element; and
a logic unit operatively coupled to the pressure sensor and the one or more temperature sensors and including logic circuitry, the logic unit configured to:
receive a value of differential pressure of the liquid from the pressure sensor and compute a value of density of the liquid;
control the heating element to apply a non-boiling heating or cooling cycle to the liquid that consumes a known amount of energy or power;
receive values of temperature of the liquid during the heating or cooling cycle from the one or more temperature sensors;
compare the temperature measurements and the value of density to a calibrated thermal model of the liquid; and
produce a value of ABV of the liquid using the comparison to the calibrated thermal model.

20. The control system of claim 19, including:
a cooling device; and
wherein the logic unit is configured to:
initiate one or more non-boiling heating and cooling cycles of a control liquid and the liquid under test;
receive values of temperature of the control liquid, the liquid under test, and a heating and cooling block containing the control liquid and liquid under test, from the one or more temperature sensors during the one or more heating and cooling cycles; and
compare the computed value of density and the temperature values of the control liquid, the liquid under test, and the heating and cooling block, to the calibrated thermal model of the liquid.

21. The control system of claim 20, wherein the logic unit is configured to:
compare the temperature measurements and the value of density to a heat transfer model of the heating and cooling block as the calibrated thermal model; and
produce a value of ABV of the liquid using the comparison to the heat transfer model.
